(19) United States
(12) Patent Application Publication
(10) Pub. No.: US 2024/0299607 A1
(43) Pub. Date: Sep. 12, 2024

(54) SYSTEM AND METHOD FOR DYNAMIC DOSING OF UV-C RADIATION

(71) Applicant: Tru-D SmartUVC, Memphis, TN (US)

(72) Inventor: Brian Manley, Memphis, TN (US)

(21) Appl. No.: 18/120,419

(22) Filed: Mar. 12, 2023

(51) Int. Cl. A61L 2/24; A61L 2/10; A61L 9/20

(52) U.S. Cl. CPC A61L 2/24; A61L 2/10; A61L 9/20; A61L 2202/11; A61L 2202/14

(57) ABSTRACT

The present disclosure relates to UV disinfection devices, systems, and methods for dynamically adjusting a power-level of each of a plurality of UV emitters of the UV disinfection device based on corresponding UV sensor measurements to minimize the total disinfection cycle time. In certain optional embodiments, the present disclosure relates to UV disinfection devices, systems, and methods for optimizing alignment of each of a plurality of UV emitters within a room to minimize the total disinfection cycle time. In other optional embodiments, the present disclosure relates to UV disinfection devices, systems, and methods for automatically preheating the plurality of UV emitters in response to the UV disinfection device being coupled to a power source, independent of a ballast (or power supply) of the plurality of UV emitters in order to minimize the total disinfection cycle time.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### FIELD OF THE INVENTION

The present disclosure relates generally to methods, systems, and devices for bacterial, fungal, and/or viral sterilization and disinfection. More particularly, the present disclosure pertains to methods, systems, and devices for sterilizing and disinfecting rooms and similar enclosed areas using radiant energy emission.

### BACKGROUND

Healthcare-Associated Infections (HAIs) are common, costly, and sometimes lethal. In American hospitals alone, the Centers for Disease Control (CDC) estimates that HAIs account for an estimated 1.7 million infections and 99,000 associated deaths each year. The nature of bacteria acquired in the hospital setting differs significantly from bacteria found in a community setting primarily in their resistance to antibiotic therapy.

Attempts to eradicate surface contaminants from the hospital setting have varied greatly in strategy and success. These have ranged from antiseptic soaps to fumigation with formaldehyde gas. Recently, ultraviolet (UV) sterilization devices have been developed and are being temporarily deployed to sterilize and disinfect entire rooms using UV-C radiant energy. Sterilization and/or disinfection of surfaces can be accomplished by illuminating or irradiating surfaces with radiant energy, such as from a UV emitting source. These types of UV disinfection (or sterilization) devices generally utilize low to medium pressure mercury lamps. More recently, amalgam lamps are being utilized as they provide up to ten times the UV power density of conventional low-pressure mercury lamps and can even be used at high ambient temperatures of up to 90° C.

One of the major issues associated with current UV disinfection devices is the amount of time it takes the device to complete a disinfection process once deployed, also known as the total disinfection cycle time (or cycle time) to achieve or accumulate a target dose of UV irradiance. The total disinfection cycle time can be critical for commercial applications of automating such disinfection. A thorough and ubiquitous treatment of the room air and surfaces may be required for a process improvement over manual methods. The total amount of UV-C that is available for irradiating an area is typically limited by the amount of power available to make UV-C from standard electrical commercial and residential building circuits. Health care facilities are generally limited to a standard 20 Amp service and other facilities may have either 15 or 20 amp service. A system that does not manage the available power will almost assuredly waste UV-C output and prolong treatment time.

Typical UV disinfection devices include a plurality of UV emitters configured to produce a stable, non-varying amount of UV energy that is equally distributed around the circumference of the UV disinfection device. These typical UV disinfection devices further feature an array (e.g., a 360 degree array) of UV sensors which measure the reflected UV irradiance from the extents of an area being disinfected. By measuring reflected UV irradiance, the device is able to determine when an adequate amount of UV energy (dose) has been delivered to an area, both in direct line of sight and in shadows. The device terminates the disinfection process by deactivating all of the UV emitters once all of the UV sensors have accumulated a predetermined amount of UV irradiance, prescribed as a target dose, which is sufficient to disinfect the area in which the device is located.

Because all sensors must achieve the prescribed target dose, total disinfection cycle time will be dictated by at least one sensor. Should one sensor measure a lower reflected irradiance than all other sensors, all emitters will remain active until this sensor achieves the target dose. Concurrently, other sensors which measure higher levels of reflected irradiance will achieve the target dose much sooner. In effect, some areas of the room will receive more UV energy than necessary while waiting on another area of the room to achieve the target dose. This inefficiency may increase the total disinfection cycle time.

Another factor affecting the total disinfection cycle time is the device placement and/or the alignment of each of the plurality of UV emitters within the room. Generally, the current UV disinfection devices, once positioned in room remain completely stationary, as to the plurality of UV emitters of the device.

### BRIEF SUMMARY

In view of at least some of the above-referenced problems in conventional UV disinfection devices an exemplary object of the present disclosure may be to provide a new and improved system and method for optimizing alignment of each of the plurality of UV emitters within a room and dynamically adjusting (dynamic dosing) a power-level of each of the plurality of UV emitters based on corresponding UV sensor measurements to minimize the total disinfection cycle time. An exemplary such system may desirably feature multiple variable-power (dimmable) UV energy emitters, allowing a UV disinfection device to adjust its UV output dynamically throughout the course of a disinfection cycle. UV sensors which measure a lower level of reflected irradiance, compared to other sensors, may have their corresponding emitter power increased such that the measured reflected irradiance increases. UV sensors which measure a higher level of reflected irradiance, compared to other sensors, may have their corresponding emitter power decreased. An exemplary such system may desirably utilize the maximum available power from a wall outlet. As a result, dynamic dosing overcomes the limitations described for current UV disinfection devices. Total disinfection cycle time may still be based on the sensor which measures the lowest level of reflected irradiance, however, dynamic dosing can increase this value such that total disinfection cycle time is reduced. An exemplary such system may feature an auto-focus feature whereby the system automatically rotates the plurality of UV emitters to an optimal position such that total disinfection cycle time is reduced.

The new and improved system and method may generally be configured to execute the following phases: deployment phase, warm-up phase, auto-focus phase, dynamic dosing phase, and shut down phase.

During the deployment phase, the operator may be instructed to setup the UV disinfection device and prepare the room for optimal disinfection. In certain optional embodiments, when the device is first energized by plugging into an available wall outlet, a flux accelerator system of the UV disinfection device may be automatically activated and may remain active until device shut down. The flux accelerator device may be configured to preheat the UV emitters in order to reduce a warm-up time thereof and therefor also decrease the total disinfection cycle time. Upon leaving the room, the operator may secure all entryways and enter operator credentials in the remote (e.g., the first step in the remote setup procedure). The operator may then be asked to confirm that the room is secure and that they are safely positioned outside the room. Upon confirmation, the warm-up phase may begin.

Upon commencement of the warm-up phase, UV emitters are activated and begin to approach maximum operational capacity. Because UV emitters are active, an in-room motion detection system of the UV disinfection device is also activated at this time. The UV disinfection device may use one or more of a warm-up timer and/or emitter-power feedback to determine when the warm-up phase is complete. Once complete, the system will automatically proceed to the auto-focus phase (optional) or proceed to the dynamic dosing phase. Remote setup procedures may or may not be complete at this time.

In an optional embodiment, during the optional auto-focus phase the device may rotate the emitter and sensor arrays to capture reflected irradiance measurements by each UV sensor in at least two array positions. By obtaining measurements in multiple positions, reflected irradiance may be captured with high resolution over a 3600 field of view. The system may then compare the irradiance measurements captured by each UV sensor in each array position, and returns the emitter and sensor arrays to an optimal position for completing the disinfection cycle. Once the emitter and sensor arrays reach the optimal position, the dynamic dose phase may begin. Remote setup procedures may or may not be complete during this phase but must be completed before proceeding too far into the next phase.

Upon commencement of the dynamic dosing phase, the system begins to vary the power-level of the UV emitters activated during the warm-up phase. While emitter power-level of the UV emitters is varied, the system may analyze the resulting impact to measured irradiance values at each UV sensor. The total disinfection cycle time impact of each UV sensor is determined, relative to measured irradiance values, and the system may determine the appropriate order in which each UV sensor will be targeted to achieve the required disinfection dose. Sensors which measure the lowest levels of reflected irradiance during the analysis period may be targeted to achieve the required dose first, while sensors measuring a higher level of reflected irradiance will be targeted last. As a result, the system may vary the power-level of each of the UV emitters to maximize the measured irradiance of each UV sensor, in order from lowest initial value to highest initial value. As such, the system may be able to achieve the fastest allowable total disinfection cycle time under limited power availability from a standard wall outlet.

Finally, in accordance with the shut down phase, once the last UV sensor within the array of UV sensors achieves the required disinfection dose, the disinfection cycle is complete. The flux accelerator system and all (or any remaining) active emitters are deactivated. At this point, the operator may be alerted of a completed disinfection cycle. Further, upon completion, all cycle statistics and device diagnostic data may be transmitted to a central database.

### BRIEF DESCRIPTION OF THE SEVERAL VIEWS OF THE DRAWINGS

FIG. 1A is a perspective view of an ultraviolet (UV) disinfection system in accordance with the present disclosure.

FIG. 1B is a partially exploded perspective view of the UV disinfection system of FIG. 1A in accordance with the present disclosure.

FIG. 1C is an exploded perspective view of the UV disinfection system of FIG. 1A in accordance with the present disclosure.

FIG. 2 is an upper perspective view of the UV disinfection system of FIG. 1A with primary and auxiliary UV disinfection devices thereof deployed in different areas in accordance with the present disclosure.

FIG. 3A is a perspective view of the primary UV disinfection device of the UV disinfection system of FIG. 1A with UV emitters of the device in a deployed position in accordance with the present disclosure.

FIG. 3B is a perspective view of the auxiliary UV disinfection device of the UV disinfection system of FIG. 1C with UV emitters of the device in a deployed position in accordance with the present disclosure.

FIG. 4 is an enlarged perspective view of the primary UV disinfection device of FIG. 3A taken of area 4 of FIG. 3A in accordance with the present disclosure.

FIG. 5 is an enlarged perspective view of the primary UV disinfection device of FIG. 3A taken of area 5 of FIG. 4 in accordance with the present disclosure.

FIG. 6 is a block diagram of an exemplary control system of the UV disinfection system of FIG. 1A in accordance with the present disclosure.

FIG. 7 is a flowchart of a method of disinfecting an area with a predetermined disinfection dose of UV energy in accordance with the present disclosure.

FIG. 8 is a flowchart containing optional method steps in accordance with the flowchart of FIG. 7 in accordance with the present disclosure.

FIG. 9A is a flowchart containing optional method steps in accordance with the flowchart of FIG. 7 in accordance with the present disclosure.

FIG. 9B is a continuation of the flowchart of FIG. 9A in accordance with the present disclosure.

FIG. 10 is a flowchart of another embodiment of a method of disinfecting an area with a predetermined disinfection dose of UV energy in accordance with the present disclosure.

### DETAILED DESCRIPTION

Reference will now be made in detail to embodiments of the present disclosure, one or more drawings of which are set forth herein. Each drawing is provided by way of explanation of the present disclosure and is not a limitation. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

Thus, it is intended that the present disclosure covers such modifications and variations as come within the scope of the appended claims and their equivalents. Other objects, features, and aspects of the present disclosure are disclosed in, or are obvious from, the following detailed description. It is to be understood by one of ordinary skill in the art that the present discussion is a description of exemplary embodiments only and is not intended as limiting the broader aspects of the present disclosure.

The words “connected”, “attached”, “joined”, “mounted”, “fastened”, and the like should be interpreted to mean any manner of joining two objects including, but not limited to, the use of any fasteners such as screws, nuts and bolts, bolts, pin and clevis, and the like allowing for a stationary, translatable, or pivotable relationship; welding of any kind such as traditional MIG welding, TIG welding, friction welding, brazing, soldering, ultrasonic welding, torch welding, inductive welding, and the like; using any resin, glue, epoxy, and the like; being integrally formed as a single part together; any mechanical fit such as a friction fit, interference fit, slidable fit, rotatable fit, pivotable fit, and the like; any combination thereof; and the like.

Unless specifically stated otherwise, any part of the apparatus of the present disclosure may be made of any appropriate or suitable material including, but not limited to, metal, alloy, polymer, polymer mixture, wood, composite, or any combination thereof.

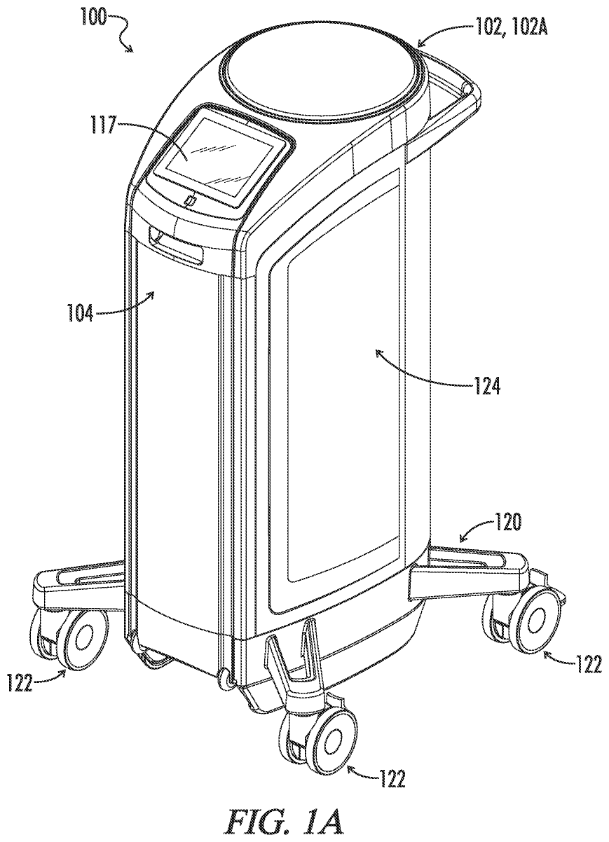

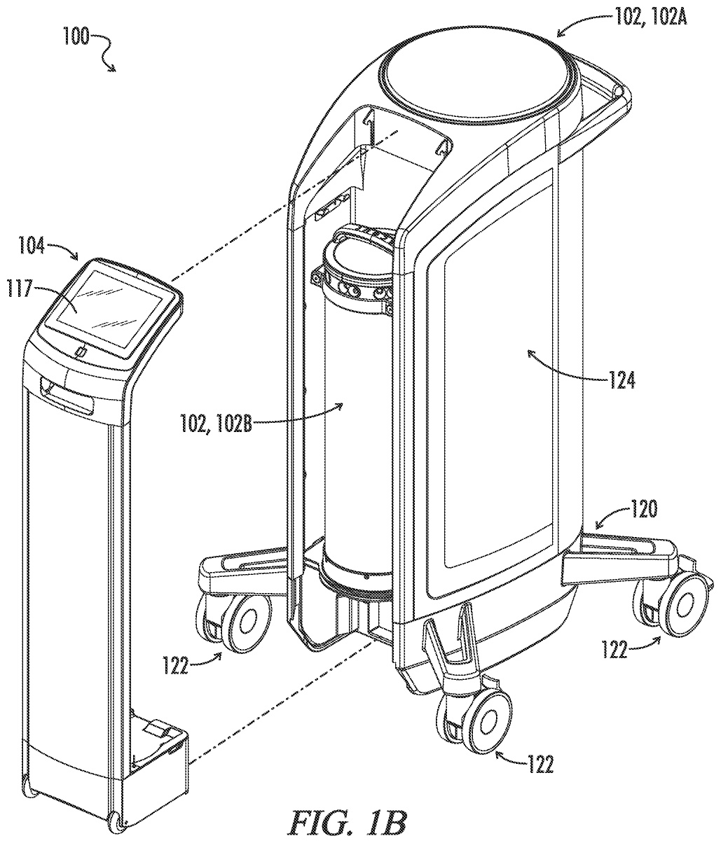

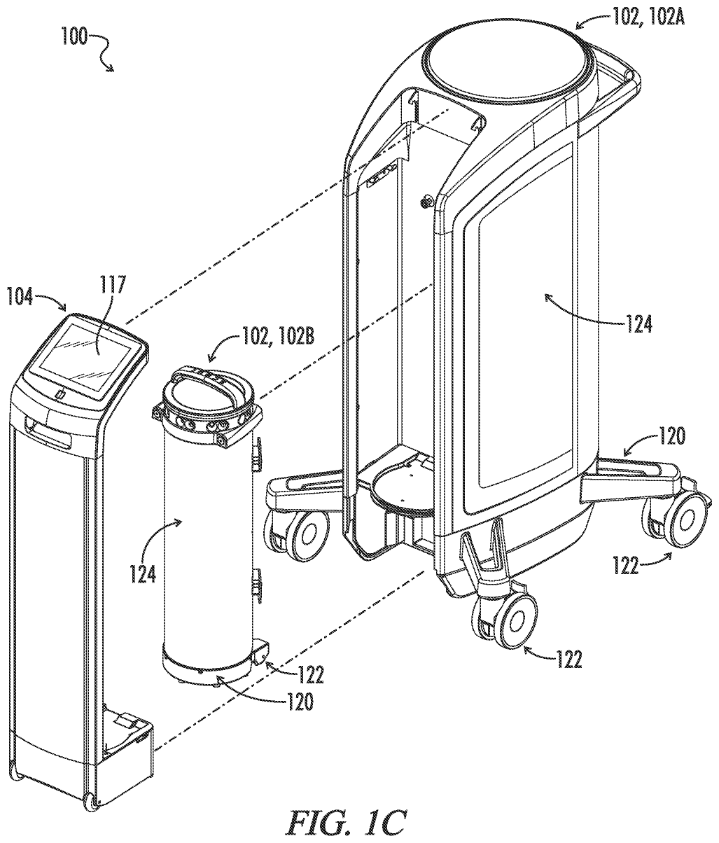

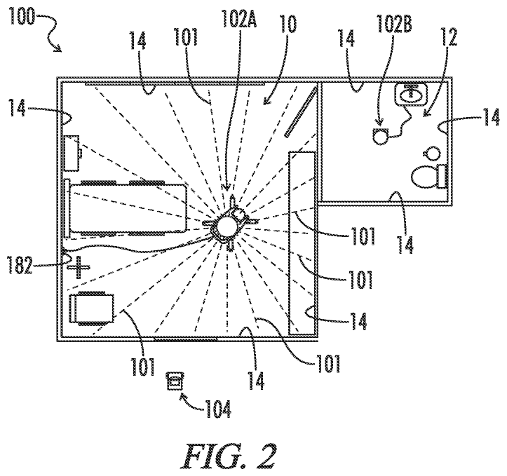

Referring to FIGS. 1A-3B, various embodiments of a disinfection system 100 for irradiating an area 10 or 12 (as illustrated in FIG. 2) with ultraviolet (UV) light are illustrated. The disinfection system 100 may include one or more UV disinfection devices 102 and a mobile control unit 104 configured to be positioned safely outside of the area 10 or 12. The mobile control unit 104 may be configured to remotely control the one or more UV disinfection devices 102. In certain optional embodiments, the mobile control unit 104 may be configured to remotely control the one or more UV disinfection devices 102, provided that only one of the one or more UV disinfection devices 102 at a time may have an active connection to the mobile control unit 104.

As illustrated in FIG. 1A, the one or more UV disinfection devices 102 of the UV disinfection system 100 may include a primary UV disinfection device 102A. The primary UV disinfection device 102A may also be referred to herein as a UV disinfection device 102A. The primary UV disinfection device 102A may be configured to carry the mobile control unit 104. The mobile control unit 104 may be configured to dock with and may even be charged by the primary UV disinfection device 102A. As illustrated in FIG. 1B, the one or more UV disinfection devices 102 of the UV disinfection system 100 may include an auxiliary UV disinfection device 102B. The auxiliary UV disinfection device 102B may be configured to be carried by the primary UV disinfection device 102A. The auxiliary UV disinfection device 102B may also be configured to dock with and may even be charged by the primary UV disinfection device 102A. As illustrated by FIG. 1C, each of the mobile control unit 104 and the auxiliary UV disinfection device 102B are exploded (or separated) from the primary UV disinfection device 102A.

As illustrated in FIG. 2, the UV disinfection system 100 may utilize the one or more UV disinfection devices 102 to disinfect one or more associated areas 10, 12 with UV light 101. The UV light 101 may also be referred to herein as UV energy 101. The auxiliary UV disinfection device 102B may be useful for cleaning small areas 12 (e.g., bathrooms or the like) while the primary UV disinfection device 102A may be more useful for cleaning large areas 10 (e.g., a patient's room, an operating room, or the like).

As disclosed herein, the auxiliary UV disinfection device 102B may be a scaled down version of the primary UV disinfection device 102A. Accordingly, elements of each of the primary UV disinfection device 102A and the auxiliary UV disinfection device 102B may be numbered and function similarly. As such, the following disclosures reference to the one or more UV disinfection devices 102 may be equally applicable to each of the primary UV disinfection device 102A and the auxiliary UV disinfection device 102B.

The one or more UV disinfection devices 102 may include a mobile base 120. The mobile base 120 may include one or more ground engaging units 122 which may be configured to support the one or more UV disinfection devices 102 and enable the mobility thereof.

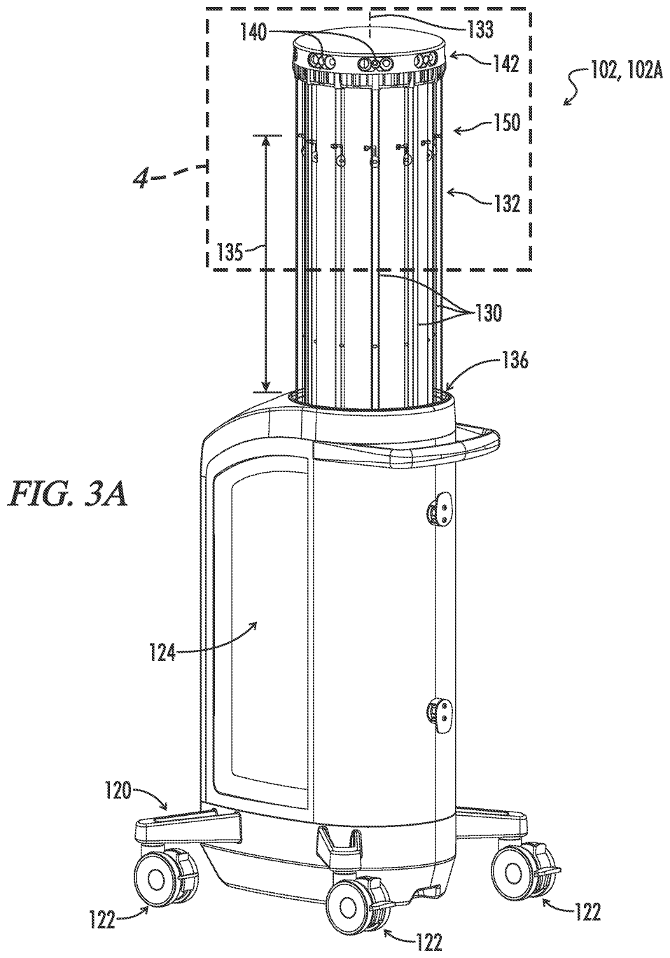

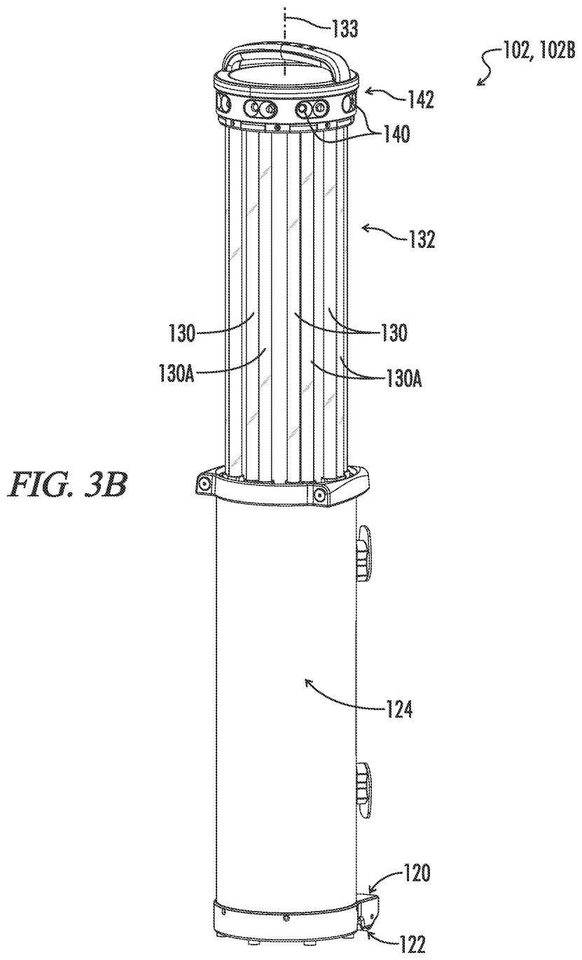

As illustrated in FIGS. 3A-5, the one or more UV disinfection devices 102 may further include a plurality of UV emitters 130 and a plurality of UV sensors 140 configured to telescope upward from within a housing 124 of the one or more UV disinfection devices 102 coupled to the mobile base 120. The plurality of UV emitters 130 may also be referred to herein as a plurality of UV lamps 130. Each of the plurality of UV emitters 130 may be tube-like, oriented vertically, and configured to emit UV light 101 (as illustrated in FIG. 2). Further, the plurality of UV emitters 130 may be positioned in an emitter array 132. The emitter array 132 may also be referred to herein as a circumferential emitter array 132. In certain optional embodiments, each of the plurality of UV emitters 130 may be associated with at least one of the plurality of UV sensors 140. In other embodiments, each of the plurality of UV emitters 130 may be vertically aligned with at least one of the plurality of UV sensors 140.

The plurality of UV sensors 140 may be positioned in a sensor array 142. The sensor array 142 may also be referred to herein as circumferential sensor array 142. In certain optional embodiments, the emitter array 132 and the sensor array 142 may be rigidly coupled together and generally referred to as an array. In other optional embodiments, each of the emitter array 132 and the sensor array 142 may be configured to rotate independently of each other.

The plurality of UV sensors 140 may be configured to generate reflected irradiance data 144 corresponding to a measured reflected irradiance value by each of the plurality of UV sensors 140. Each of the plurality of UV sensors 140 may be narrowly focused and configured to measure only reflected irradiance so as to exclude measuring direct radiant energy from each of the plurality of UV emitters 130. Accordingly, each of the plurality of UV sensors 140 is configured to only measure the radiant energy as reflected (e.g., reflected irradiant energy) from a specific point of one of the one or more surfaces 14 of the area 10 or 12. The reflected irradiance data 144 may include: (1) a plurality of initial reflected irradiance values measured by each of the plurality of UV sensors 140 and corresponding to a plurality of array positions of the circumferential sensor array 142 (wherein the plurality of array positions may be rotationally offset from each other), (2) optimal position initial reflected irradiance values measured by the plurality of UV sensors 140 corresponding to an optimal array position, (3) real-time reflected irradiance values measured before, during, or after the power-level of each of the plurality of UV emitters 130 is dynamically adjusted, or (4) the like.

Each of the plurality of UV emitters 130 may be any type of lamp or bulb configured to emit UVC energy, such as, for example, a low-pressure mercury lamp, an amalgam lamp, or the like. Each of the plurality of UV emitters 130 may be adjustable (e.g., dimmable) based upon a power-level supplied to each of the plurality of UV emitters 130. The power-level supplied to each of the plurality of UV emitters 130 may be at least partially controlled by at least one ballast 138. The at least one ballast 138 may also be referred to herein as at least one power supply 138.

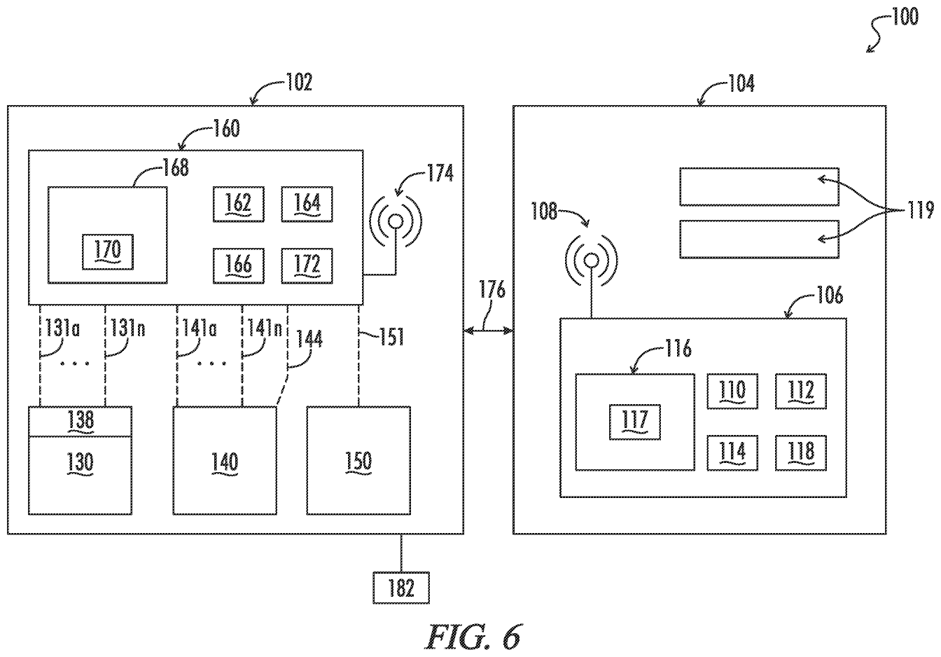

Referring to FIG. 6, an exemplary control system of the UV disinfection system 100 is schematically illustrated. The one or more UV disinfection devices 102 may include a single controller 160 or discrete controllers for each of at least the plurality of UV emitters 130, the plurality of UV sensors 140, and the auxiliary amalgam heating assembly 150 (when present). Similarly, the mobile control unit 104 may include a single controller 106 or discrete controllers for controlling at least one of the primary UV disinfection device 102A and the auxiliary UV disinfection device 102B.

The controller 160 of the one or more UV disinfection devices 102 may include a processor 162, a computer readable memory medium 164, a database 166, and an input/output module or control panel 168 having a display 170. Likewise, the controller 106 of the mobile control unit 104 may include a processor 110, a computer readable memory medium 112, a database 114, and an input/output module or control panel 116 having a display 117.

As illustrated, the controller 160 of the one or more UV disinfection devices 102 may be configured to receive reflected irradiance data 144 from each of the plurality of UV sensors 140. The controller 160 may also receive various other inputs from internal and external sources regarding other operating parameters of the UV disinfection device 102, such as, for example, whether movement is sensed within the area 10 or 12, whether a door to the area 10 or 12 is secured, or the like. In other optional embodiments (not shown), the controller 106 of the mobile control unit 104 may be configured to receive this same data remotely from the one or more UV disinfection devices 102, for example, using a wireless transceiver 108 coupled to the controller 106 and a wireless transceiver 174 coupled to the controller 160. Each of the wireless transceivers 108, 174 may be configured to utilize radio frequency (RF), Bluetooth, Bluetooth Low Energy, WiFi, cellular, pulse light, or the like, to send and receive data 176.

Based upon various operational parameters which may be defined by the computer programming product 172, the controller 160 may generate various control signals which may be communicated to each of the plurality of UV emitters 130 (schematically illustrated via the dashed communication lines 131a-n, where n is the number of UV emitters), each of the plurality of UV sensors 140 (schematically illustrated via the dashed communication lines 141a-n, where n is the number of UV sensors), and the auxiliary amalgam heating assembly 150 (schematically illustrated via the dashed communication line 151). In certain optional embodiments, based upon various operational parameters which may be defined by the computer programming product 118, the controller 106 may generate various control signals which may be communicated to the one or more UV disinfection devices 102.

Each of the primary UV disinfection device 102A, the auxiliary UV disinfection device 102B, and the mobile control unit 104 may be powered by an external power source 182, such as, for example, alternating current (AC). In certain optional embodiments, the mobile control unit 104 may further include a power source 119, such as, for example, one or more batteries. In certain optional embodiments, the power source 119 may be charged by plugging the mobile control unit 104 into an external power source 182. In other optional embodiments, the power source 119 may be charged by the primary UV disinfection device 102A when the mobile control unit 104 is docked with the primary UV disinfection device 102A and the primary UV disinfection device 102A is plugged in. The external power source 182 may generally be limited to a twenty (20) ampere service.

Because the total amount of power which may be utilized by the one or more UV disinfection devices 102 is typically limited by the external power source 182, it is an objective of the UV disinfection system 100 to efficiently and effectively allocate power between each of the plurality of UV emitters 130 and reduce a treatment time to complete a disinfection cycle 190. Another object of the UV disinfection system 100 is, regardless of a positioned of the primary UV disinfection device 102A with the area 10 to optimally align the plurality of UV emitters 130 within the area 10 to reduce the treatment time to complete the disinfection cycle 190.

The control system, via one or more of the controllers 106, 160, may be configured to rotate at least the emitter array 132 to an optimal array position based at least in part on a plurality of initial reflected irradiance values of the reflected irradiance data 144 of the plurality of UV sensors 140 associated with a plurality of initial array positions. The plurality of initial array positions may be rotationally offset from each other.

The control system, via one or more of the controllers 106, 160, may further be configured to dynamically adjust a power-level of each of the plurality of UV emitters 130 and receive the reflected irradiance data 144 corresponding to each adjusted power-level for maximizing the reflected irradiance data 144 in order of priority from lowest to highest of optimal reflected irradiance values of the reflected irradiance data 144 corresponding to the optimal array position.

The control system, via one or more of the controllers 106, 160, may further be configured begin a disinfection cycle 190 and track a total accumulated UV irradiance by each of the plurality of UV sensors 140. The total accumulated UV irradiance depending at least in part on a sensed reflected irradiance value of each of the plurality of UV sensors 140 and an elapsed time since beginning the disinfection cycle 190.

The control system, via one or more of the controllers 106, 160, may further be configured to complete the disinfection cycle 190 once the total accumulated UV irradiance of each of the plurality of UV sensors 140 is greater than or equal to a predetermined disinfection dose. The predetermined disinfection dose may also be referred to herein as a germicidal dose, a bactericidal dose, or the like.

In certain optional embodiments, the control system, via one or more of the controllers 106, 160, may further be configured to: (i) reduce the power-level of each of the plurality of UV emitters 130 by a predetermined percentage, (ii) identify a lowest initial value of the reflected irradiance values corresponding to the optimal array position and associated with a lowest sensor of the plurality of UV sensors, (iii) maximize a sensed reflected irradiance value of the lowest sensor by increasing the power-level of a corresponding at least one the plurality of UV emitters associated with the lowest sensor of the plurality of UV sensors, (iv) define a target reflected irradiance value corresponding to the maximized sensed reflected irradiance value of the lowest sensor, and (v) sequentially increase the power-level of remaining ones of the plurality of UV emitters 130 beginning with a next lowest initial value of the reflected irradiance values corresponding to the optimal array position until each sensed reflected irradiance value corresponding to each of the plurality of UV sensors 140 is greater than or equal to the target reflected irradiance value.

In certain optional embodiments, the control system, via one or more of the controllers 106, 160, may further be configured to selectively deactivate each of the plurality of UV emitters 130 once the total accumulated UV irradiance of a corresponding sensor of the plurality of UV sensors 140 is greater than or equal to the predetermined disinfection dose. In accordance with this embodiment, the control system, via one or more of the controllers 106, 160, may further be configured to dynamically vary the power-level of remaining active emitters of the plurality of UV emitters 130 for maximizing the sensed reflected irradiance values in order of priority from lowest to highest of the sensed reflected irradiance values of certain ones of the plurality of UV sensors 140 associated with the remaining active emitters.

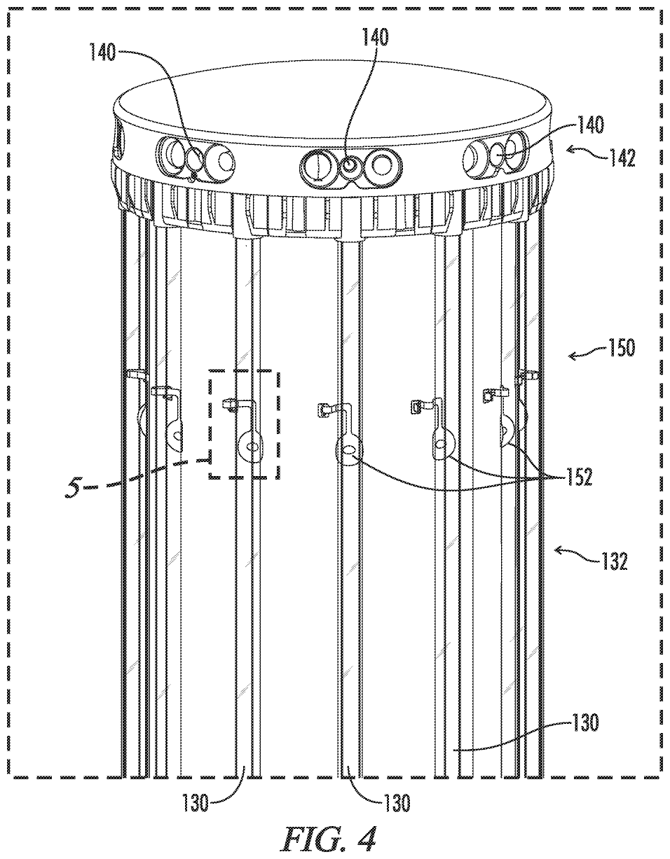

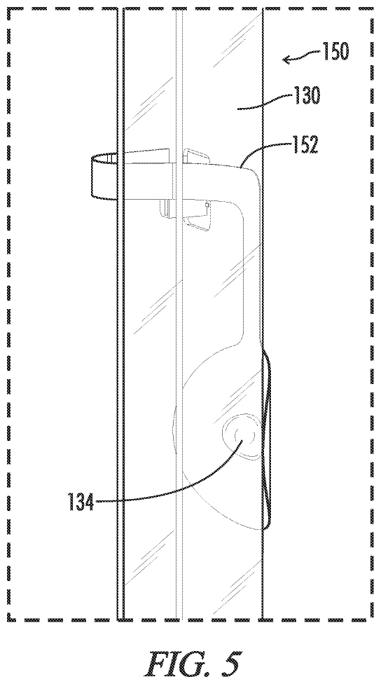

In certain optional embodiments, the control system, via one or more of the controllers 106, 160, may be configured to automatically in response to power being applied to the one or more UV disinfection devices 102, preheat the plurality of UV emitters 130 using an auxiliary amalgam heating assembly 150 having a plurality of heating elements 152 positioned proximate to each of the plurality of UV emitters 130. The auxiliary amalgam heating assembly 150 may also be referred to herein as a flux accelerator assembly 150 or a flux accelerator apparatus 150. In accordance with this embodiment, and as illustrated in FIGS. 4-5, each of the plurality of UV emitters 130 may be an amalgam lamp having an amalgam spot 134 coupled to an interior surface of the lamp. Accordingly, and in accordance with this embodiment, the control system, via one or more of the controllers 106, 160, may be configured to automatically activate the auxiliary amalgam heating assembly 150 to heat the amalgam spot 134 of each of the plurality of UV emitters 130 in response to a power state of the one or more UV disinfection devices 102. The auxiliary amalgam heating assembly 150 may operate independently of the ballast 138 of the plurality of UV emitters 130. By automatically activating the auxiliary amalgam heating assembly 150, a preheating time of each of the plurality of UV emitters 130 may be reduced as the auxiliary amalgam heating assembly 150 is activated before the plurality of UV emitters 130 are activated.

The amalgam spot 134 of each of the plurality of UV emitters 130 may be positioned at a common height 135 relative to a lower end 136 of each of the plurality of UV emitters 130. In certain optional embodiments, the amalgam spot 134 of each of the plurality of UV emitters 130 may be positioned such that they are proximate to a central axis 133 of the emitter array 132. In other words, each of the plurality of UV emitters 130 may be rotated such that the amalgam spot 134 of each of the plurality of UV emitters 130 is as close as possible to the central axis 133.

The power state of the one or more UV disinfection devices 102 may correspond to whether the device is coupled to the external power source 182, such as, for example, AC power. In certain optional embodiments, the power state may also or alternatively correspond to whether the one or more UV disinfection devices 102 is in an on state or an off state.

The terms “controller,” “control circuit” and “control circuitry” as used herein may refer to, be embodied by or otherwise included within a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed and programmed to perform or cause the performance of the functions described herein. A general purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration.

The term “computer-readable memory medium” as used herein may refer to any non-transitory medium 112, 164 alone or as one of a plurality of non-transitory memory media 112, 164 within which is embodied in a computer program product 118, 172 that includes processor-executable software, instructions or program modules which upon execution may provide data or otherwise cause a computer system to implement subject matter or otherwise operate in a specific manner as further defined herein. It may further be understood that more than one type of memory media may be used in combination to conduct processor-executable software, instructions or program modules from a first memory medium upon which the software, instructions or program modules initially reside to a processor for execution.

“Memory media” as generally used herein may further include without limitation transmission media and/or storage media. “Storage media” may refer in an equivalent manner to volatile and non-volatile, removable and non-removable media, including at least dynamic memory, application specific integrated circuits (ASIC), chip memory devices, optical or magnetic disk memory devices, flash memory devices, or any other medium which may be used to stored data in a processor-accessible manner, and may unless otherwise stated either reside on a single computing platform or be distributed across a plurality of such platforms. “Transmission media” may include any tangible media effective to permit processor-executable software, instructions or program modules residing on the media to be read and executed by a processor, including without limitation wire, cable, fiber-optic and wireless media such as is known in the art.

The term “processor” as used herein may refer to at least general-purpose or specific-purpose processing devices and/or logic as may be understood by one of skill in the art, including but not limited to single- or multithreading processors, central processors, parent processors, graphical processors, media processors, and the like.

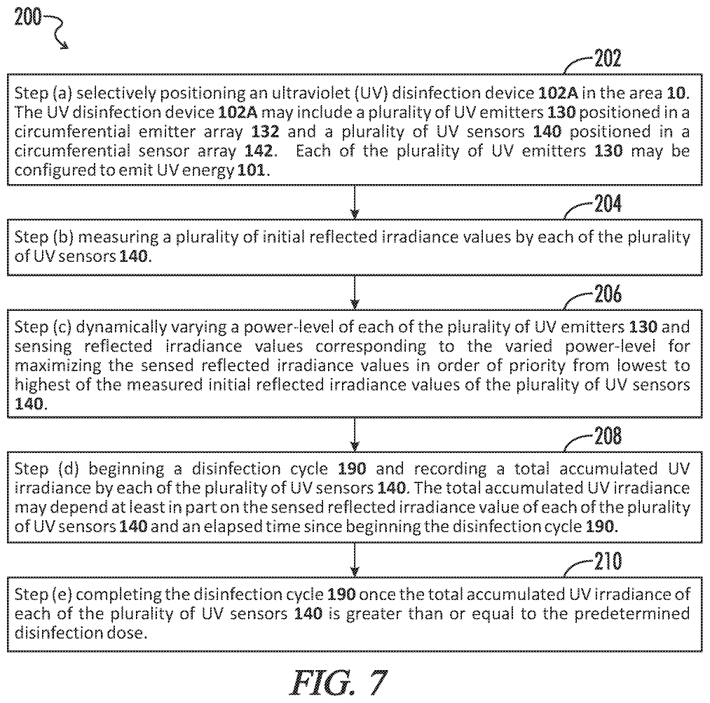

Referring to FIG. 7, with further illustrative reference back to FIGS. 1A-6, an embodiment of a method 200 may now be described which is exemplary but not limiting on the scope the present disclosure unless otherwise specifically noted. One of skill in the art may appreciate that alternative embodiments may include fewer or additional steps, and that certain disclosed steps may for example be performed in different chronological order or simultaneously. Unless otherwise specifically noted, operations, steps, functions, processes, and the like as disclosed in association with the method 200 may be executed or directed by a single computing device, or via multiple computing devices in operable communication via a communications network. Exemplary such computing devices may include onboard controllers 106, 160 or machine control systems, remote (e.g., cloud) servers, mobile user devices, and the like.

The method 200 may be directed to disinfecting an area 10 or 12 with a predetermined disinfection dose of UV light 101. The method 200 may include step (a) selectively positioning 202 an ultraviolet (UV) disinfection device 102A in the area 10. As discussed above, the primary UV disinfection device 102A may include a plurality of UV emitters 130 positioned in a circumferential emitter array 132 and a plurality of UV sensors 140 positioned in a circumferential sensor array 142. Each of the plurality of UV emitters 130 may be configured to emit UV light 101.

The method 200 may further include step (b) measuring 204 a plurality of initial reflected irradiance values by the plurality of UV sensors 140. In certain optional embodiments, for example when an autofocus feature is implemented, the plurality of initial reflected irradiance values may be measured by each of the plurality of UV sensors 140 and may corresponding to a plurality of array positions of the circumferential sensor array 142. The plurality of array positions may be rotationally offset. In certain optional embodiments, the both the circumferential sensor array 142 and the circumferential emitter array 132 may rotated in unison to each of the plurality of array positions. In other optional embodiments, the circumferential sensor array 142 may rotationally move to each of the plurality of array positions while the circumferential emitter array 132 remains stationary.

The method 200 may further include step (c) dynamically varying 206 a power-level of each of the plurality of UV emitters 130 and sensing reflected irradiance values corresponding to the varied power-level for maximizing the sensed reflected irradiance values in order of priority from lowest to highest of the measured optimal position initial reflected irradiance values of the plurality of UV sensors 140.

The method 200 may further include step (d) beginning 208 a disinfection cycle 190 and recording a total accumulated UV irradiance by each of the plurality of UV sensors 140. The total accumulated UV irradiance may depend at least in part on the sensed reflected irradiance value of each of the plurality of UV sensors 140 and an elapsed time since beginning the disinfection cycle 190.

The method 200 may further include step (e) completing 210 the disinfection cycle 190 once the total accumulated UV irradiance of each of the plurality of UV sensors 140 is greater than or equal to the predetermined disinfection dose.

In further optional embodiments, the method 200 may further comprise, prior to step (b), warming-up the plurality of UV emitters 130 until a ballast current of each of the plurality of UV emitters 130 is stabilized.

In certain optional embodiments, the method 200 may further include, following step (b) rotating the circumferential emitter array 132 to an optimal array position. The optimal array position may be determined at least in part on at least one of the measured plurality of initial reflected irradiance values of the plurality of UV sensors 140. Following this step, the method 200 may further include measuring an optimal position initial reflected irradiance value by each of the plurality of UV sensors 140 corresponding to the optimal array position. In certain optional embodiments, the step of measuring the optimal position initial reflected irradiance values may be performed with each of the plurality of UV emitters 130 set at a maximum power level or a predetermined percentage of the maximum power level. In certain optional embodiments, the circumferential emitter array 132 and the circumferential sensor array 142 may be configured to simultaneously move, such as, for example, to maintain alignment of each of the plurality of UV sensors 140 with each of the plurality of UV emitters 130.

In certain optional embodiments, for example, when the autofocus feature is implemented, step (c) of the method 200 may prioritize each of the plurality of UV emitters 130 for maximizing the sensed reflected irradiance values in order of priority from lowest to highest of the measured optimal position initial reflected irradiance values of the plurality of UV sensors 140.

In other optional embodiments, the method 200 may further comprise, prior to rotating the circumferential emitter array to the optimal array position, comparing each of the plurality of initial reflected irradiance values measured by each of the plurality of UV sensors 140 corresponding to each of the plurality of array positions of the circumferential sensor array 142, and determining the optimal array position of the circumferential emitter array 132 as one of the plurality of array positions or between any two of the plurality of array positions.

In certain optional embodiments, the step of determining the optimal array position of the method 200 may further comprise: identifying a lowest measured minimum value of the plurality of initial reflected irradiance values of the plurality of UV sensors 140 corresponding to a lowest minimum array position of the plurality array positions; and defining the optimal array position as the lowest minimum array position. This optional method step may depend upon the efficacy impact of rotating the plurality of UV sensors 140. If efficacy varies greatly with sensor rotation, then the plurality of UV sensors 140 may be placed in a non-optimal position. This may be similar to the current method of operation which uses the lowest sensor to determine the total disinfection cycle time. By rotating sensors to find the lowest of the lows (e.g., the lowest measured minimum value), adequate disinfection dosage in the entire treatment area may be guaranteed.

In other optional embodiments, the step of determining the optimal array position of the method 200 may further comprise: identifying a lowest measured minimum value of the plurality of initial reflected irradiance values corresponding to one of the plurality of array positions; identifying a highest measured minimum value of the plurality of initial reflected irradiance values corresponding to a different one of the plurality of array positions; identifying a midpoint array position of the plurality of array positions wherein each of the plurality of initial reflected irradiance values of the plurality of UV sensors 140 corresponding to the midpoint array position is greater than or equal to an average between the lowest measured minimum value and the highest measured minimum value of the plurality of initial reflected irradiance values of the plurality of UV sensors 140; and defining the optimal array position as the identified midpoint array position. This optional method step may depend upon the efficacy impact of rotating the plurality of UV sensors 140. If efficacy varies minimally with sensor rotation, then the plurality of UV sensors 140 may be rotated such that the lowest sensor value represents a mid-point between the highest measured minimum value (e.g., optimal reading) and the lowest measured minimum value (non-optimal reading). This position may seek to reduce the total disinfection cycle time while minimizing impacts to efficacy.

In further optional embodiments, the step of determining the optimal array position of the method 200 may further comprise: identifying a highest measured minimum value of the plurality of initial reflected irradiance values corresponding to a highest minimum array position the plurality of array positions; and defining the optimal array position as the highest minimum array position. This optional method step may depend upon the efficacy impact of rotating the plurality of UV sensors 140. If sensor rotation has no impact on efficacy, then the plurality of UV sensors 140 may be rotated in such a way to maximize the reading from the lowest sensor (e.g., the highest measured minimum value). This may reduce the total disinfection cycle time as much as possible without manually repositioning the device.

In certain optional embodiments, step (c) of the method 200 may further comprise: optionally reducing the power-level of each of the plurality of UV emitters by a predetermined percentage (e.g., if not previously reduced in step (b)); identifying a lowest initial value of the optimal position initial reflected irradiance values associated with a lowest sensor of the plurality of UV sensors 140; maximizing the sensed reflected irradiance value of the lowest sensor by increasing the power-level of a corresponding one the plurality of UV emitters 130 associated with the lowest sensor of the plurality of UV sensors 140; and defining a target reflected irradiance value corresponding to the maximized sensed reflected irradiance value of the lowest sensor.

In other optional embodiments, the method 200 may further comprise increasing the power-level of two of the plurality of UV emitters 130 adjacent to the corresponding one of the plurality of UV emitters associated with the lowest sensor of the plurality of UV sensors 140 prior to defining the target reflected irradiance value.

In further optional embodiments, the method 200 may further comprise sequentially increasing the power-level of remaining ones of the plurality of UV emitters 130 beginning with a next lowest initial value of the initial reflected irradiance values until each of the sensed reflected irradiance values is greater than or equal to the target reflected irradiance value. In certain optional embodiments, this step may be performed with a next lowest initial value of the optimal position initial reflected irradiance values.

In still further optional embodiments, the method 200 may further comprise activating one or more additional UV emitters 130A (shown in FIG. 3B) to increase the sensed reflected irradiance values of each of the plurality of UV sensors 140 to be greater than or equal to the target reflected irradiance value. In certain optional embodiments, the one or more additional UV emitters 130A may be positioned between or next to each of the plurality of UV emitters 130. In other optional embodiments, the one or more additional UV emitters 130A may be positioned between selected ones of the plurality of UV emitters 130.

In certain optional embodiments, step (a) of the method 200 may further comprise: applying power to the UV disinfection device 102; and preheating the plurality of UV emitters 130 automatically in response to the applied power using a using a flux accelerator apparatus 150 having one or more heating elements 152 positioned proximate to each of the plurality of UV emitters 130.

In other optional embodiments, step (e) of the method 200 may further comprise selectively deactivating each of the plurality of UV emitters 130 once the total accumulated UV irradiance of a corresponding sensor of the plurality of UV sensors 140 is greater than or equal to the predetermined disinfection dose.

In further optional embodiments, the method 200 may further comprise dynamically varying the power-level of remaining active emitters of the plurality of UV emitters 130 for maximizing the sensed reflected irradiance values in order of priority from lowest to highest of the sensed reflected irradiance values of certain ones of the plurality of UV sensors 140 associated with the remaining active emitters.

In other optional embodiments, rather than selectively deactivating each of the plurality of UV emitters 130 once the total accumulated UV irradiance of a corresponding sensor of the plurality of UV sensors 140 is greater than or equal to the predetermined disinfection dose, those specific UV emitters may be dimmed such that power directed to them may be redirected to other UV emitters (e.g., in progress UV emitters) of the plurality of UV emitters 130 which have not achieved the total accumulated UV irradiance. The redirected power may be prioritized as disclosed above, for example, to in progress UV emitters in order of priority from lowest to highest of the sensed reflected irradiance values of certain ones of the plurality of UV sensors 140 associated with the in progress UV emitters.

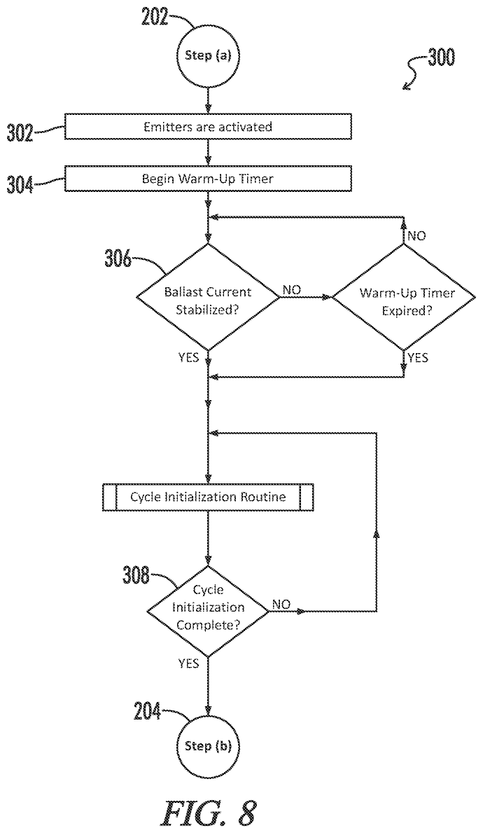

Referring to FIG. 8, an optional method step flowchart 300 illustrating additional optional details of the method 200 which may occur between steps (b) and (c) is provided. In certain optional embodiments, the method 200 may further include activating 302 the plurality of UV emitters 130, while the one or more additional UV emitters 130A remain inactive. In other optional embodiments, the method 200 may further include beginning 304 a warm-up timer and monitoring 306 the ballast current. Once either the ballast current has stabilized or the warm-up timer has expired, the method 200 may further include confirming 308 whether a cycle initialization is complete. In certain optional embodiments, the method 300 may further include determining whether a remote setup is confirmed and/or confirming whether a door monitor is active. The remote setup may referred to a setup of the mobile control unit 104.

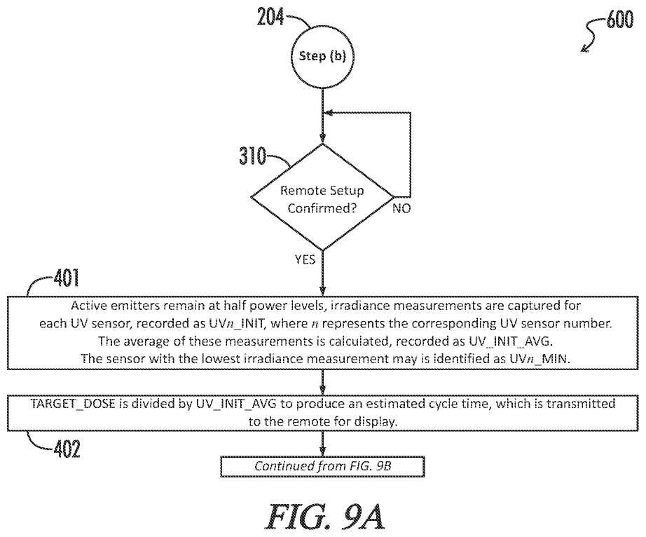

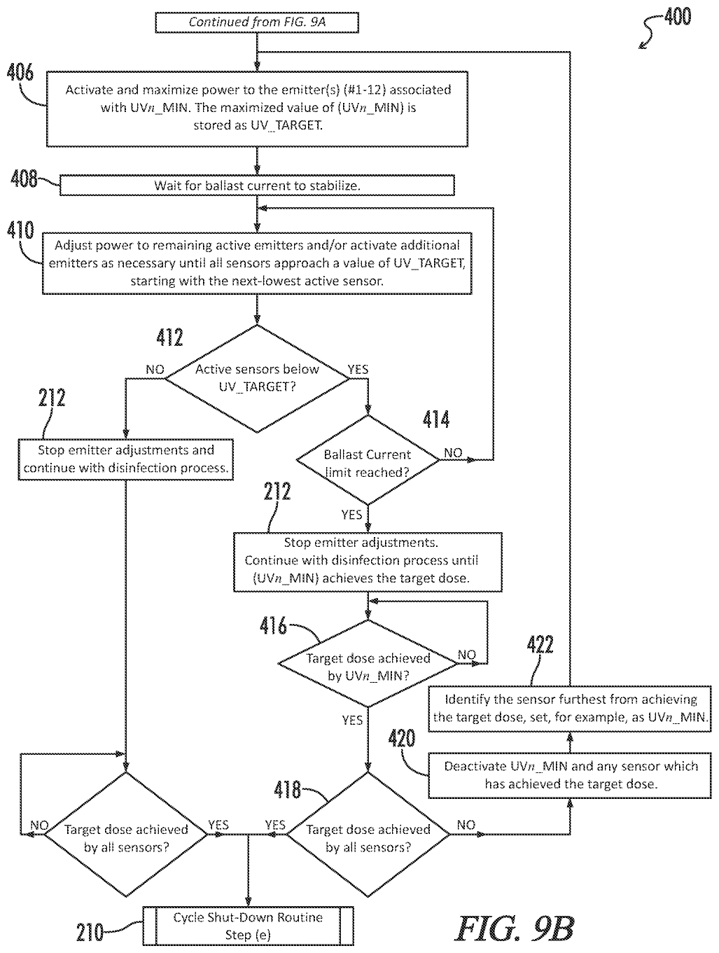

Referring to FIGS. 9A-9B, an optional method step flowchart 400 illustrating additional optional details of the method 200 which may occur in or between any of steps (b), (c), (d), or (e) is provided, but are generally performed during step (c) (e.g., the dynamic dosing phase). In certain optional embodiments, determining 310 whether the remote setup is confirmed may be performed during any of steps (b), (c), or (d) of the method 200 as illustrated by the optional method step flowchart 400.

In certain optional embodiments, the optional method step flowchart 400 may include providing 401 a predetermined percentage of the maximum power to the plurality of UV emitters 130, measuring the reflected irradiance for each of the plurality of UV sensors 140, averaging these measurements, and determining the lowest measured reflected irradiance associated with one of the plurality of UV sensors 130. The optional method step flowchart 400 may further include calculating 402 an estimated total disinfection cycle time by dividing the predetermined disinfection dose by the average reflected irradiance. The method 400 may further include transmitting the estimated total disinfection cycle time to the mobile control unit 104.

In certain optional embodiments, 401 of the optional method step flowchart 400 may be performed at maximum power and may further utilize optimal position initial reflected irradiance value by each of the plurality of UV sensors 140. Following this, the optional method step flowchart 400 may include reducing the power-level of each of the plurality of UV emitters 130 by a predetermined percentage. The predetermined percentage may for example be approximately 50% of the maximum power level.

Next, the method 200 may further include maximizing 406 a power-level of one of the plurality of emitters 130 associated with the lowest optimal sensor, while also activating and/or maximizing a power-level of at least one emitter adjacent thereto. In certain optional embodiments, the at least one emitter adjacent thereto may be two emitters adjacent on opposite sides thereto. The at least one emitter may either be of the plurality of UV emitters 130 or of the one or more additional UV emitters 130A. A maximized value of the lowest optimal sensor may be stored as a target reflected irradiance value. Following the maximizing 406 step, the method 200 may include waiting 408 for the ballast current to stabilize.

In certain optional embodiments, the method 200 may further include adjusting 410 a power level to remaining active emitters of the plurality of UV emitters 130 and/or activating one or more additional UV emitters 130A, as necessary, until the sensed reflected irradiance value of each of the plurality of UV sensors 140 approaches (e.g., is greater than or equal to) the target reflected irradiance value. This step may be sequentially performed based upon a next lowest sensor of the plurality of UV sensors 140.

As illustrated by the optional method step flowchart 400 in FIG. 9B, the method 200 may optionally further include determining 412 whether any of the plurality of sensors 140 is below the target reflected irradiance value. If none of the plurality of UV sensors 140 is below the target reflected irradiance value, then the method 200 may proceed with step (d) until the predetermined disinfection dose has been achieved by all of the plurality of UV sensors 140. If any of the plurality of UV sensors 140 is below the target reflected irradiance value, then the method 200 may further include determining 414 whether a maximum allowable ballast current has been reached. The maximum allowable ballast current may depend upon a total available power supply provided by the external power source 182. If the maximum allowable ballast current has not been reached, then the method 200 will revert back to continuing adjusting 410 the power level to certain active emitters of the plurality of UV emitters 130 associated with any of the plurality of UV sensors 140 measuring below the target reflected irradiance value.

If the maximum allowable ballast current has been reached, then the method 200 may proceed with step (d) until the predetermined disinfection dose. In certain optional embodiments, at least point, the method 200 may further include determining 416 whether the predetermined disinfection dose has been achieved by the lowest optimal sensor and further determining 418 whether the predetermined disinfection dose has been achieved by all of the plurality of UV sensors 140.

If the predetermined disinfection dose has not been achieved by all of the plurality of UV sensors 140, then the method 200 may further include deactivating 420 any of the plurality of UV sensors 140 which have achieved the predetermined disinfection dose. In certain optional embodiments, the step of deactivating 420 may further include deactivating any of the plurality of UV emitters 130 associated with the deactivated UV sensors. Next, the method 200 may optionally include identifying 422 a sensor of the plurality of UV sensors 140 furthest from achieving the predetermined disinfection dose and then proceeding to repeat steps 406, 408, 410, etc. for remaining active UV emitters associated with that sensor and any other remaining active sensors, sequentially from lowest to highest reflected irradiance value.

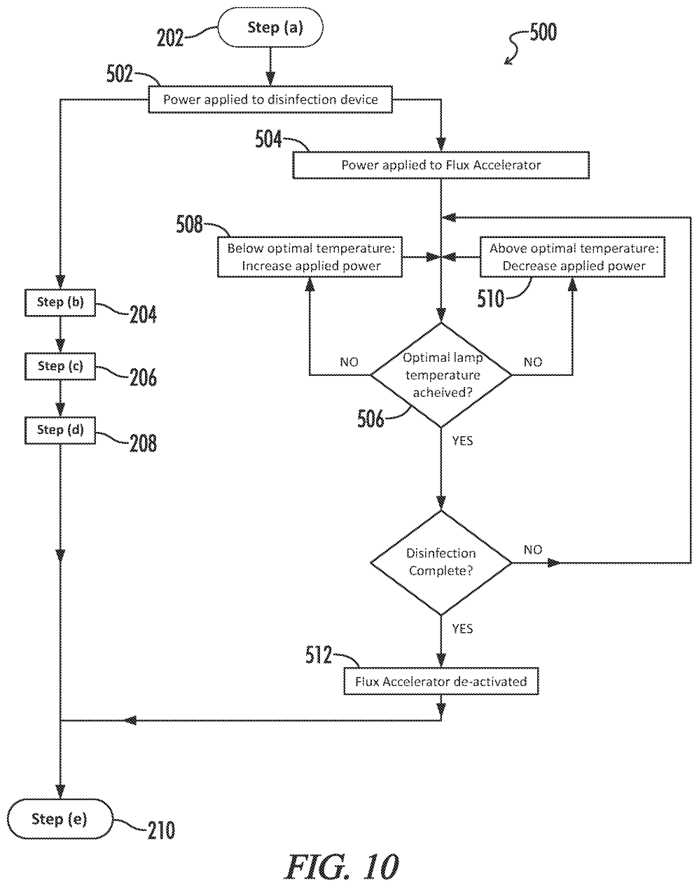

Referring to FIG. 10, an embodiment of a method 500 of disinfecting an area 10 or 12 with a predetermined disinfection dose of UV energy 101. The method 500 may include step (a) of the method 200. The method 500 may further include applying 502 power (e.g., from an external power source 182) to the primary UV disinfection device 102A. The method 500 may further include automatically in response to the applied power, activating 504 the auxiliary amalgam heating assembly 150 to heat the amalgam spot 134 of each of the plurality to UV emitters 130. The method 500 may further include step (b), step (c), step (d), and step (e) of the method 200 being performed independently of (e.g., in parallel with) operation of the auxiliary amalgam heating assembly 150.

The method 500 may further include monitoring 506 a temperature of the amalgam spot 134 of at least one of the plurality of UV emitters 130. The method 500 may further include increasing 508 an applied power-level to the auxiliary amalgam heating assembly 150 when the monitored temperature is below a predetermined optimal emitter temperature. The method 500 may further include decreasing 510 the applied power-level to the auxiliary amalgam heating assembly 150 when the monitored temperature is above the predetermined optimal emitter temperature. In certain optional embodiments, the applied power-level to each of the plurality of heating elements 154 of the auxiliary amalgam heating assembly 150 may be controlled simultaneously. In other optional embodiments, the applied power-level to each of the plurality of heating elements 154 of the auxiliary amalgam heating assembly 150 may be independently sensed and controlled.

Once the disinfection cycle 190 is complete, the method 500 may further include deactivating 512 the auxiliary amalgam heating assembly 150. The method 500 may end with step (e) of the method 200. In certain optional embodiments, step (e) of the method 200 may include deactivating 512 the auxiliary amalgam heating assembly 150.

Throughout the specification and claims, the following terms take at least the meanings explicitly associated herein, unless the context dictates otherwise. The meanings identified below do not necessarily limit the terms, but merely provide illustrative examples for the terms. The meaning of “a,” “an,” and “the” may include plural references, and the meaning of “in” may include “in” and “on.” The phrase “in one embodiment,” as used herein does not necessarily refer to the same embodiment, although it may.

Although embodiments of the present invention have been described in detail, it will be understood by those skilled in the art that various modifications can be made therein without departing from the spirit and scope of the invention as set forth in the appended claims.

This written description uses examples to disclose the invention and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It will be understood that the particular embodiments described herein are shown by way of illustration and not as limitations of the invention. The principal features of this invention may be employed in various embodiments without departing from the scope of the invention. Those of ordinary skill in the art will recognize numerous equivalents to the specific procedures described herein. Such equivalents are considered to be within the scope of this invention and are covered by the claims.

All of the compositions and/or methods disclosed and claimed herein may be made and/or executed without undue experimentation in light of the present disclosure. While the compositions and methods of this invention have been described in terms of the embodiments included herein, it will be apparent to those of ordinary skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

The previous detailed description has been provided for the purposes of illustration and description. Thus, although there have been described particular embodiments of a new and useful invention, it is not intended that such references be construed as limitations upon the scope of this disclosure except as set forth in the following claims.

What is claimed is:

1. A method of disinfecting an area with a predetermined disinfecting dose of UV energy, the method comprising:
(a) selectively positioning an ultraviolet (UV) disinfection device in the area, the UV disinfection device including a plurality of UV emitters positioned in a circumferential emitter array and a plurality of UV sensors positioned in a circumferential sensor array, each of the plurality of UV emitters configured to emit UV energy;
(b) measuring a plurality of initial reflected irradiance values by the plurality of UV sensors;
(c) dynamically varying a power-level of each of the plurality of UV emitters and sensing reflected irradiance values corresponding to the varied power-level for maximizing the sensed reflected irradiance values in order of priority from lowest to highest of the initial reflected irradiance values of the plurality of UV sensors;
(d) beginning a disinfection cycle and recording a total accumulated UV irradiance by each of the plurality of UV sensors, the total accumulated UV irradiance depending at least in part on the sensed reflected irradiance value of each of the plurality of UV sensors and an elapsed time since beginning the disinfection cycle; and
(e) completing the disinfection cycle once the total accumulated UV irradiance of each of the plurality of UV sensors is greater than or equal to the predetermined disinfecting dose.

2. The method of claim 1, wherein step (e) further comprises:
selectively deactivating each of the plurality of UV emitters once the total accumulated UV irradiance of a corresponding sensor of the plurality of UV sensors is greater than or equal to the predetermined disinfecting dose.

3. The method of claim 2, further comprising:
dynamically varying the power-level of remaining active emitters of the plurality of UV emitters for maximizing the sensed reflected irradiance values in order of priority from lowest to highest of the sensed reflected irradiance values of certain ones of the plurality of UV sensors associated with the remaining active emitters.

4. The method of claim 1, wherein:
each of the plurality of UV emitters is vertically aligned with at least one of the plurality of UV sensors.

5. The method of claim 1, further comprising prior to step (b):
warming-up the plurality of UV emitters until a ballast current of each of the plurality of UV emitters is stabilized.

6. The method of claim 1, further comprising between steps (b) and (c):
associating the plurality of initial reflected irradiance values with corresponding array positions of the circumferential sensor array, the plurality of array positions being rotationally offset;
rotating the circumferential emitter array to an optimal array position; and
repeating step (b) with the circumferential emitter array in the optimal array position.

7. The method of claim 6, further comprising prior to rotating the circumferential emitter array to an optimal array position:
comparing each of the plurality of initial reflected irradiance values measured by each of the plurality of UV sensors corresponding to each of the plurality of array positions of the circumferential sensor array; and
determining the optimal array position of the circumferential emitter array as at one of the plurality of array positions or between any two of the plurality of array positions.

8. The method of claim 7, wherein the step of determining the optimal array position further comprises:
identifying a lowest measured minimum value of the plurality of initial reflected irradiance values of the plurality of UV sensors corresponding to a lowest minimum array position of the plurality array positions; and
defining the optimal array position as the lowest minimum array position.

9. The method of claim 7, wherein the step of determining the optimal array position further comprises:
identifying a lowest measured minimum value of the plurality of initial reflected irradiance values corresponding to one of the plurality of array positions;
identifying a highest measured minimum value of the plurality of initial reflected irradiance values corresponding to a different one of the plurality of array positions;
identifying a midpoint array position of the plurality of array positions wherein each of the plurality of initial reflected irradiance values of the plurality of UV sensors corresponding to the midpoint array position is greater than or equal to an average between the lowest measured minimum value and the highest measured minimum value of the plurality of initial reflected irradiance values of the plurality of UV sensors; and
defining the optimal array position as the identified midpoint array position.

10. The method of claim 7, wherein the step of determining the optimal array position further comprises:
identifying a highest measured minimum value of the plurality of initial reflected irradiance values corresponding to a highest minimum array position the plurality of array positions; and
defining the optimal array position as the highest minimum array position.

11. The method of claim 1, wherein step (c) further comprises:
reducing the power-level of each of the plurality of UV emitters by a predetermined percentage;
identifying a lowest initial value of the initial reflected irradiance values associated with a lowest sensor of the plurality of UV sensors;
maximizing the sensed reflected irradiance value of the lowest sensor by increasing the power-level of a corresponding one the plurality of UV emitters associated with the lowest sensor of the plurality of UV sensors; and
defining a target reflected irradiance value corresponding to the maximized sensed reflected irradiance value of the lowest sensor.

12. The method of claim 11, further comprising increasing the power-level of two of the plurality of UV emitters adjacent to the corresponding one of the plurality of UV emitters associated with the lowest sensor of the plurality of UV sensors prior to defining the target reflected irradiance value.

13. The method of claim 11, further comprising:
sequentially increasing the power-level of remaining ones of the plurality of UV emitters beginning with a next lowest initial value of the initial reflected irradiance values until each of the sensed reflected irradiance values is greater than or equal to the target reflected irradiance value.

14. The method of claim 13, further comprising:
activating one or more additional UV emitters to increase the sensed reflected irradiance values of each of the plurality of UV sensors to be greater than or equal to the target reflected irradiance value.

15. The method of claim 1, wherein step (a) further comprises:
applying power to the UV disinfection device; and
preheating the plurality of UV emitters automatically in response to the applied power using a using a flux accelerator apparatus having one or more heating elements positioned proximate to each of the plurality of UV emitters.

16. A disinfection system for disinfecting an area, the disinfection system comprising:
a mobile ultraviolet (UV) disinfection device including a plurality of UV sensors and a plurality of UV emitters positioned in an array, each of the plurality of UV emitters corresponding to at least one of the plurality of UV sensors, each of the plurality of UV sensors configured to generate reflected irradiance data corresponding to a measured reflected irradiance received indirectly from one or more of the plurality of UV emitters via reflection off of one or more surfaces of the area; and
a controller linked to receive the reflected irradiance data from the plurality of UV sensors and configured to:
dynamically adjust a power-level of each of the plurality of UV emitters and receive the reflected irradiance data corresponding to each adjusted power-level for maximizing the reflected irradiance data in order of priority from lowest to highest of reflected irradiance data;
begin a disinfection cycle and track a total accumulated UV irradiance by each of the plurality of UV sensors, the total accumulated UV irradiance depending at least in part on a sensed reflected irradiance value of each of the plurality of UV sensors and an elapsed time since beginning the disinfection cycle; and
complete the disinfection cycle once the total accumulated UV irradiance of each of the plurality of UV sensors is greater than or equal to a predetermined disinfecting dose.

17. The disinfection system of claim 16, wherein the controller is configured to:
reduce the power-level of each of the plurality of UV emitters by a predetermined percentage;
identify a lowest initial value of the reflected irradiance values associated with a lowest sensor of the plurality of UV sensors;
maximize a sensed reflected irradiance value of the lowest sensor by increasing the power-level of a corresponding at least one the plurality of UV emitters associated with the lowest sensor of the plurality of UV sensors;
define a target reflected irradiance value corresponding to the maximized sensed reflected irradiance value of the lowest sensor; and
sequentially increase the power-level of remaining ones of the plurality of UV emitters beginning with a next lowest initial value of the reflected irradiance values corresponding to the optimal array position until each sensed reflected irradiance value corresponding to each of the plurality of UV sensors is greater than or equal to the target reflected irradiance value.

18. The disinfection system of claim 16, wherein the controller is configured to:
rotate the array to an optimal array position based at least in part on a plurality of initial reflected irradiance values of the plurality of UV sensors associated with a plurality of initial array positions, plurality of initial array positions may be rotationally offset from each other;
reduce the power-level of each of the plurality of UV emitters by a predetermined percentage;
identify a lowest initial value of the reflected irradiance values corresponding to the optimal array position and associated with a lowest sensor of the plurality of UV sensors;
maximize a sensed reflected irradiance value of the lowest sensor by increasing the power-level of a corresponding at least one the plurality of UV emitters associated with the lowest sensor of the plurality of UV sensors;
define a target reflected irradiance value corresponding to the maximized sensed reflected irradiance value of the lowest sensor; and
sequentially increase the power-level of remaining ones of the plurality of UV emitters beginning with a next lowest initial value of the reflected irradiance values corresponding to the optimal array position until each sensed reflected irradiance value corresponding to each of the plurality of UV sensors is greater than or equal to the target reflected irradiance value.

19. The disinfection system of claim 16, wherein the controller is configured to:
selectively deactivate each of the plurality of UV emitters once the total accumulated UV irradiance of a corresponding sensor of the plurality of UV sensors is greater than or equal to the predetermined disinfecting dose; and
dynamically vary the power-level of remaining active emitters of the plurality of UV emitters for maximizing the sensed reflected irradiance values in order of priority from lowest to highest of the sensed reflected irradiance values of certain ones of the plurality of UV sensors associated with the remaining active emitters.

20. The disinfection system of claim 16, wherein the controller is configured to:
automatically in response to power being applied to the UV disinfection device, preheat the plurality of UV emitters using a using a flux accelerator apparatus having one or more heating elements positioned proximate to each of the plurality of UV emitters.
